(19) United States
(12) Patent Application Publication
(10) Pub. No.: US 2006/0141512 A1
(43) Pub. Date: Jun. 29, 2006

(54) Novel method for separation of human sperm from biological samples for application in human identification

(76) Inventors: Sudhir K. Sinha, New Orleans, LA (US); Jaiprakash G. Shewale, New Orleans, LA (US)

Correspondence Address: Robert E. Bushnell, Suite 300, 1522 K Street, N.W., Washington, DC, 20005, US

(21) Appl. No.: 11/292,286

(22) Filed: Dec. 2, 2005

Related U.S. Application Data
(60) Provisional application No. 60/632,600, filed on Dec. 3, 2004.

(51) Int. Cl. C12Q 1/68

(52) U.S. Cl. 435/6; 435/325

(57) ABSTRACT

The development of an isolation methodology for separation of human sperm cells from biological samples containing human epithelial cells is provided. Using sperm binding proteins, glycopeptides, lectins, derivatives of N-acetylglucosamine, triazine dyes or inhibitors of glycosyltransferase linked to an insoluble support, our invention enables binding of human sperm cells from biological samples. Bound sperms can be dissociated and used for in vitro analyses or subsequently lysed on the insoluble support for isolation of male specific DNA. Other cell types such as epithelial cells, white blood cells and cell debris present in the biological samples are not bound to the derivatized insoluble support. The sperm cells, thus isolated, can be processed for isolation of nuclear DNA for human identification and forensic DNA analysis.

### CLAIM OF PRIORITY

This application makes reference to, incorporates the same herein, and claims all benefits accruing under 35 U.S.C. §119 from a provisional application entitled A Novel Method for Separation of Human Sperm from Biological Samples for Application in Human Identification filed in the United States Patent & Trademark Office on Dec. 3, 2004, and there duly assigned Ser. No. 60/632,600 by that Office.

### BACKGROUND OF INVENTION

1. Field of Invention

The present invention relates to binding of sperm cells via macromolecular interactions to macromolecules linked to an insoluble support and more particularly to separation of sperm cells from biological samples containing a mixture of cells or cell extracts.

2. Description of the Related Art

A typical evidence sample from a rape or sexual assault case contains mixture of sperm cells from the assailant and epithelial cells from the victim. Separation and isolation of DNA from sperm and epithelial cells are critical and limiting steps in identification of an assailant. Genotypes for the victim and contributor(s) are obtained by amplification of respective DNA preparations for highly polymorphic short tandem repeats (STRs) by the polymerase chain reaction (PCR). The most widely used method for selective isolation of DNA from epithelial and sperm cells is differential extraction (See Gill P, Jeffreys A J, Werret D J. Forensic application of DNA ‘fingerprints’. Nature 1985; 318: 577-579; Yoshida K, Sekiguchi K, Mizuno N, Kasai K, Sakai I, Sato H, Seta S. The modified method of two-step differential extraction of sperm and vaginal epithelial cell DNA from vaginal fluid mixed with semen. Forensic Sci Int. 1995; 72: 25-33; and Budowle B, Smith J, Moretti T, DiZinno J. DNA typing protocols: Molecular biology and forensic analysis. 2000; Eaton Publishing, Natik, Mass., USA). This approach involves digestion of epithelial cells by proteinase K in the presence of sodium sodecyl sulphate (SDS), separation of sperm cells from epithelial cell fraction by centrifugation, and digestion of sperm cells by proteinase K in the presence of dithiothreitol (DTT) to obtain the sperm cell fraction. DNA from the epithelial and sperm cell fractions is isolated by using standard known procedures. This approach suffers from several disadvantages such as the presence of sperm cell DNA in the epithelial cell fraction due to sperm degradation during processing, presence of residual epithelial cells in the sperm cells fraction, loss of sperm cells during the operations, and tedious operation. Several other approaches have been explored for separation of sperm and epithelial cell fractions. These include laser microdissection (see Elliott K, Hills D S, Lambert C, Burroughes T R, Gill P. Use of laser microdissection greatly improves the recovery of DNA from sperm on microscope slides. Forensic Sci Int. 2003; 137: 28-36), flow cytometry (see Di Nunno N, Melato M, Vimercati A, Di Nunno C, Costantinides F, Vecchiotti C, Frezzini C, Cina S, Vimercati F. DNA identification of sperm cells collected and stored by flow cytometry. Am J Forensic Med Pathol. 2003; 24: 254-270), immunomagnetic cell-separation (see Hoffstat D V, Deforce D, Van den Eekhout E. Isolation of specific cells from forensic samples for human DNA typing. 12^{th} International Symposium on Human Identification. 2001, Biloxi, Miss., USA), use of filers with controlled pore size (see Chen J, Kobilinsky L, Wolosin D, Shaler R, Baum H. A physical method for separating spermatozoa from epithelial cells in sexual assault evidence. J Forensic Sci. 1998; 43: 114-118; Garvin A M. Filtration based DNA separation for sexual assault cases. J Forensic Sci. 2003; 48: 1084-1087), and microdevice (see Isolation of sperm cells from other cells in cell mixtures, http://www.uvapf.org/technologies/index.cfin/fuseaction/invention/invention_id/180/). None of these methods, however, have replaced the differential extraction procedure as a routine laboratory practice.

### SUMMARY OF INVENTION

It is therefore an object of the present invention to provide an apparatus for separation of sperm cells from biological samples.

It is also an object of the present invention to provide a method for highly selective isolation of human sperm cells from a biological sample using macromolecules present on the surface of egg cells

It is another object of the present invention to provide a method for highly selective isolation of human sperm cells from a biological sample using macromolecular interactions involved in binding of sperm to the structural moieties analogous to the moieties present on the surface of egg cells.

It is yet another object of the present invention to provide a method, which is useful for processing of many samples simultaneously, either manually or on a robotic platform.

It is yet another object of the present invention to provide an affinity isolation methodology for separation of human sperm cells from biological samples containing human epithelial cells, treated and untreated.

Using sperm binding proteins, glycopeptides, lectins, derivatives of N-acetylglucosamine or triazine dyes linked to an insoluble support, our invention enables binding of human sperm cells from biological samples. Bound sperms can be dissociated and used for in vitro analyses or subsequently lysed on the insoluble support for isolation of male specific DNA. Other cell types such as epithelial cells, white blood cells and cell debris present in the biological samples are not bound to the derivatized insoluble support. The sperm cells, thus isolated, can be processed for isolation of nuclear DNA for human identification and forensic analysis.

Attachment of zona pellucida (ZP) proteins, glycopeptides, lectins, derivatives of N-acetylglucosamine or triazine dyes to an insoluble support such as magnetic beads makes our methodology high throughput and automation compatible for processing of many samples simultaneously. Mainstream application of this methodology will undoubtedly benefit human identification, resolving rape (sexual assault) case evidence samples and forensic DNA analysis.

### DESCRIPTION OF THE PREFERRED EMBODIMENTS

To overcome the limitations associated with previously reported methods for isolation of sperm cells, we have used a systematic approach for the rapid isolation of sperm cells from biological samples. These include, but are not limited to, binding of sperm cells to ZP proteins, glycopeptides, lectins, structural analogues of N-acetylglucosamine or triazine dyes linked to an insoluble support.

Sperm binds to egg via interactions between protein molecules located on the surface of the sperm and the ZP region of the egg (see Vo L H, Hedrick J L. Independent and hetero-oligomeric-dependent sperm binding to egg envelope glycoprotein ZPC in Xenopus laevis. Biol Reprod. 2000; 62: 766-774; Howes L, Jones R. Interactions between zona pellucida glycoproteins and sperm proacrosin/acrosin during fertilization. J Reprod Immunol. 2002; 53: 181-192; Okumura H, Kohno Y, Iwata Y, Mori H, Aoki N, Sato C, Kitajima K, Nadano D, Matsuda T. A newly identified zona pellucida glycoprotein, ZPD, and dimeric ZP1 of chicken egg envelope are involved in sperm activation on sperm-egg interaction. Biochem J. 2004; 384: 191-199; Vo L H, Yen T-Y, Macher B A, Hedrick J L. Identification of the ZPC oligosaccharide ligand involved in sperm binding and the glycan structures of Xenopus laevis vitelline envelope glycoproteins. Biol Reprod. 2003; 69: 1822-1830). The ZP region of egg is comprised of many sperm binding proteins.

N-acteylglucosamine-Sepharose medium enables isolation of galactosyltransferase enzyme from biological fluids (Daruwalla K R, Nakhasi H L, Qasba P K. Galactosyltransferase purified from rat milk is distinct from the human and bovine enzyme. J Dairy Sci. 1986; 69: 1806-1810). Similarly, triazine dyes like Cibacron Blue 3GA, Reactive Red 120, Reactive Yellow 86, Reactive Green 19, Reactive Blue 4 and Reactive Brown 10 inhibit the glycosyltransferase by interactions with the sugar binding sites (Kaminska J, Dzeciol J, Koscielak J. Triazine dyes as inhibitors and affinity ligands of glycosyltransferase. Gluconjugate J. 1999; 16: 719-723). Glycosyltransferase is isolated by using Cibacron Blue 3GA-Agarose medium (Kaminska J, Dzeciol J, Koscielak J. Triazine dyes as inhibitors and affinity ligands of glycosyltransferase. Gluconjugate J. 1999; 16: 719-723).

The sperm cell surface proteins like galactosyltransferase exhibit interactions with ZP proteins, glycopeptides, lectins, and structural analogues of N-acetylglucosamine.

The ZP proteins, synthetic glycopeptides, lectins, structural analogues of N-acetylglucosamine and triazine dyes can be linked to magnetic beads and other insoluble supports.

ZP proteins, glycopeptides, lectins, derivatives of N-acetylglucosamine or triazine dyes are linked to insoluble support via chemical linkage between the functional groups present on these moieties and reactive groups on the insoluble supports. Insoluble supports or magnetic beads with different reactive groups such as amino, carboxyl, aldehyde or epoxy are useful supports. Anchoring of the ZP proteins, glycopeptides, lectins, derivatives of N-acetylglucosamine or triazine dyes to insoluble supports with epoxy or aldehyde functional group is a single step process and does not need activation. Insoluble supports possessing carboxyl functional group need activation with chemical reagents like carbodiamide. A bi-functional reagent like glutaraldehyde is required for cross-linking the ZP proteins, glycopeptides, lectins or derivatives of N-acetylglucosamine to insoluble support with amino functional groups.

The derivatized insoluble supports thus obtained bind human sperm cells from biological samples. Sperm cells are selectively bound to the derivatized insoluble support in presence of salts and at a pH range favorable for macromolecular interactions. The insoluble nature of the derivatized support enables easier separation, for example, by physical means, of sperm cells from the biological samples containing other cells such as epithelial cells and cell debris.

Sperm cells may be eluted from the insoluble support in presence of molecules that disrupt the molecular interactions between the macromolecules on the sperm cell surface and ZP proteins, glycopeptides, lectins, structural analogues of N-acetylglucosamine or triazine dyes. Dissociated sperm cells and insoluble support can be separated by physical means. Sperm cells thus obtained are processed for extraction of DNA. Alternatively, the sperm cells bound to the insoluble support may be lysed and the male DNA isolated. The sperm cells, either separated or bound to the insoluble support, are digested with proteinase K in presence of DTT. Liberated DNA is isolated by capture on ion exchange supports, phenol-chloroform extraction, magnetic beads, ion charged supports or by using standard procedures.

The novel method described here enables separation of human sperm cells from biological samples including other types of cells, such as epithelial cells. Sperm cells thus separated can be used for different analyses, including but not limited to isolation of DNA for human identification and forensic DNA analysis. Using this method it is possible to isolate the DNA from sperm and epithelial cells for application in human identification and forensic DNA analysis.

Now, the present invention will be described in further detail with reference to Examples. However, it should be understood that the following examples are provided for a better understanding of the present invention and the present invention is by no means restricted to such specific Examples.

Cibacron Blue 3GA Agarose was equilibrated with cacodylate buffer of neutral pH containing salts such as MgCl₂ and polyhydroxy alcohol such as glycerol. The sperm suspension is mixed with the equilibrated Ciacron Blue 3GA Agarose under similar conditions used for equilibration. The suspension is incubated with intermittent stirring for a period of about 30 minutes at room temperature. Biological materials other than sperm cells were removed after incubation by washing the Ciacron Blue 3GA Agarose with cacodylate buffer of neutral pH containing salts such as MgCl₂ and polyhydroxy alcohol such as glycerol followed by Tris-HCl buffer of alkaline pH between 7.5 and 8.5 containing N-acetylglucosamine and ethylenediaminetetraacetic acid. The binding of sperm cells was tracked by microscopic observation. Most of the sperm cells remain bound to Ciacron Blue 3GA Agarose. The human male DNA from the sperm cells bound to the Ciacron Blue 3GA Agarose is isolated by digestion of the sperm cells and purification of DNA using standard procedures.

Reactive Red 120 Agarose was equilibrated with cacodylate buffer of neutral pH containing salts such as MgCl₂ and polyhydroxy alcohol such as glycerol. The sperm suspension is mixed with the equilibrated Reactive Red 120 Agarose under similar conditions used for equilibration. The suspension is incubated with intermittent stirring for a period of about 30 minutes at room temperature. Biological materials other than sperm cells were removed after incubation by washing the Reactive Red 120 Agarose with cacodylate buffer of neutral pH containing salts such as MgCl₂ and polyhydroxy alcohol such as glycerol followed by Tris-HCl buffer of alkaline pH between 7.5 and 8.5 containing N-acetylglucosamine and ethylenediaminetetraacetic acid. The binding of sperm cells was tracked by microscopic observation. Most of the sperm cells remain bound to Reactive Red 120 Agarose. The human male DNA from the sperm cells bound to the Reactive Red 120 Agarose is isolated by digestion of the sperm cells and purification of DNA using standard procedures.

N-Acetyl-D-glucosamine Agarose was equilibrated with cacodylate buffer of neutral pH containing salts such as MgCl₂ and polyhydroxy alcohol such as glycerol. The sperm suspension is mixed with the equilibrated N-Acetyl-D-glucosamine Agarose under similar conditions used for equilibration. The suspension is incubated with intermittent stirring for a period of about 30 minutes at room temperature. Biological materials other than sperm cells were removed after incubation by washing the N-Acetyl-D-glucosamine Agarose with cacodylate buffer of neutral pH containing salts such as MgCl₂ and polyhydroxy alcohol such as glycerol followed by Tris-HCl buffer of alkaline pH between 7.5 and 8.5 containing N-acetylglucosamine and ethylenediaminetetraacetic acid. The binding of sperm cells was tracked by microscopic observation. Most of the sperm cells remain bound to N-Acetyl-D-glucosamine Agarose. The human male DNA from the sperm cells bound to the N-Acetyl-D-glucosamine Agarose is isolated by digestion of the sperm cells and purification of DNA using standard procedures.

N-acetyl-D-glucosamine is dissolved in sodium phosphate buffer of alkaline pH between 8.0 and 9.0. Magnetic beads with epoxy functional groups are washed with phosphate buffer of alkaline pH between 8.0 and 9.0. Washed epoxy magnetic beads are added to the solution of N-acetyl-D-glucosamine and incubated with intermittent shaking for a period of 18 to 20 hours at room temperature. The magnetic beads derivatized with N-acetyl-D-glucosamine are washed with water followed by cacodylate buffer of neutral pH containing salts such as MgCl₂ and polyhydroxy alcohol such as glycerol. The derivatized magnetic beads have been used for isolation of sperm by the method described earlier in the embodiments.

Cibacron Blue 3GA is dissolved in sodium phosphate buffer of alkaline pH between 8.0 and 9.0. Magnetic beads with epoxy functional groups are washed with phosphate buffer of alkaline pH between 8.0 and 9.0. Washed epoxy magnetic beads are added to the solution of Cibacron Blue 3GA and incubated with intermittent shaking for a period of 18 to 20 hours at room temperature. The magnetic beads derivatized with Cibacron Blue 3GA are washed with water followed by cacodylate buffer of neutral pH containing salts such as MgCl₂ and polyhydroxy alcohol such as glycerol. The derivatized magnetic beads have been used for isolation of sperm by the method described earlier in the embodiments.

Reactive Red 120 is dissolved in sodium phosphate buffer of alkaline pH between 8.0 and 9.0. Magnetic beads with epoxy functional groups are washed with phosphate buffer of alkaline pH between 8.0 and 9.0. Washed epoxy magnetic beads are added to the solution of Reactive Red 120 and incubated with intermittent shaking for a period of 18 to 20 hours at room temperature. The magnetic beads derivatized with Reactive Red 120 are washed with water followed by cacodylate buffer of neutral pH containing salts such as MgCl₂ and polyhydroxy alcohol such as glycerol. The derivatized magnetic beads have been used for isolation of sperm by the method described earlier in the embodiments.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus for binding sperm cells, comprising:
an insoluble support; and
a compound linked to said insoluble support, said compound being at least one selected from the group consisting of zona pellucida proteins, glycopeptide and lectin.

2. The apparatus of claim 1, wherein said compound is linked to said insoluble support via chemical linkage between a reactive group of said compound and a functional group of said insoluble support.

3. The apparatus of claim 1, wherein said insoluble support has at least one functional group selected from the group consisting of amino, carboxyl, aldehyde and epoxy.

4. The apparatus of claim 1, wherein said insoluble support is magnetic beads.

5. A method for separating sperm cells from an unknown sample, the method utilizing the apparatus of claim 1.

6. An apparatus for binding sperm cells, comprising:
an insoluble support; and
a compound linked to said insoluble support, said compound being N-acetylglucosamine or its derivative.

7. The apparatus of claim 6, wherein said compound is linked to said insoluble support via chemical linkage between a reactive group of said compound and a functional group of said insoluble support.

8. The apparatus of claim 6, wherein said insoluble support has at least one functional group selected from the group consisting of amino, carboxyl, aldehyde and epoxy.

9. The apparatus of claim 6, wherein said insoluble support is magnetic beads.

10. A method for separating sperm cells from an unknown sample, the method utilizing the apparatus of claim 6.

11. An apparatus for binding sperm cells, comprising:
an insoluble support; and
a compound linked to said insoluble support, said compound being at least one selected from the Cibacron Blue 3GA, Reactive Red 120 and inhibitor of glycosyltransferase.

12. The apparatus of claim 11, wherein said compound is linked to said insoluble support via chemical linkage between a reactive group of said compound and a functional group of said insoluble support.

13. The apparatus of claim 11, wherein said insoluble support has at least one functional group selected from the group consisting of amino, carboxyl, aldehyde and epoxy.

14. The apparatus of claim 11, wherein said insoluble support is magnetic beads.

15. A method for separating sperm cells from an unknown sample, the method utilizing the apparatus of claim 11.

16. A method of separating sperm cells from an unknown sample, comprising:
selectively binding said sperm cells to an apparatus which is comprised of an insoluble support and a compound linked to said insoluble support, said compound being at least one selected from the group consisting of zona pellucida proteins, glycopeptide and lectin.

17. The method of claim 16, wherein said compound is linked to said insoluble support via chemical linkage between a reactive group of said compound and a functional group of said insoluble support.

18. The method of claim 16, wherein said insoluble support has at least one functional group selected from the group consisting of amino, carboxyl, aldehyde, and epoxy.

19. The method of claim 16, wherein said insoluble support is magnetic beads.

20. The method of claim 16, further comprising the step of separating the sperm cells from the insoluble support by physical means.

21. The method of claim 16, further comprising the step of eluting said sperm cells from the insoluble support in presence of molecules for disrupting the molecular interaction between the macromolecules on the surfaces of the sperm cells and said compound.

22. The method of claim 16, further comprising the step of lysing the sperm cells bound to insoluble support for isolation of male specific DNA.

23. A method of separating sperm cells from an unknown sample, comprising:
selectively binding said sperm cells to an apparatus which is comprised of an insoluble support and a compound linked to said insoluble support, said compound being N-acetylglucosamine or its derivative.

24. The method of claim 23, wherein said compound is linked to said insoluble support via chemical linkage between a reactive group of said compound and a functional group of said insoluble support.

25. The method of claim 23, wherein said insoluble support has at least one functional group selected from the group consisting of amino, carboxyl, aldehyde, and epoxy.

26. The method of claim 23, wherein said insoluble support is magnetic beads.

27. The method of claim 23, further comprising the step of separating the sperm cells from the insoluble support by physical means.

28. The method of claim 23, further comprising the step of eluting said sperm cells from the insoluble support in presence of molecules for disrupting the molecular interaction between the macromolecules on the surfaces of the sperm cells and said compound.

29. The method of claim 23, further comprising the step of lysing the sperm cells bound to insoluble support for isolation of male specific DNA.

30. A method of separating sperm cells from an unknown sample, comprising:
selectively binding said sperm cells to an apparatus which is comprised of an insoluble support and a compound linked to said insoluble support, said compound being at least one selected from the Cibacron Blue 3GA, Reactive Red 120 and inhibitor of glycosyltransferase.

31. The method of claim 30, wherein said compound is linked to said insoluble support via chemical linkage between a reactive group of said compound and a functional group of said insoluble support.

32. The method of claim 30, wherein said insoluble support has at least one functional group selected from the group consisting of amino, carboxyl, aldehyde, and epoxy.

33. The method of claim 30, wherein said insoluble support is magnetic beads.

34. The method of claim 30, further comprising the step of separating the sperm cells from the insoluble support by physical means.

35. The method of claim 30, further comprising the step of eluting said sperm cells from the insoluble support in presence of molecules for disrupting the molecular interaction between the macromolecules on the surfaces of the sperm cells and said compound.

36. The method of claim 30, further comprising the step of lysing the sperm cells bound to insoluble support for isolation of male specific DNA.
